(19) United States
(12) Patent Application Publication
(10) Pub. No.: US 2022/0325331 A1
(43) Pub. Date: Oct. 13, 2022

(54) SYSTEMS AND METHODS FOR EFFECTIVE DIAGNOSTIC OLIGONUCLEOTIDE DETECTION

(71) Applicant: CIBUS BIOTECHNOLOGIES, INC., Salt Lake City, UT (US)

(72) Inventors: Deric Holden, Salt Lake City, UT (US); Eve F. Fabrizio, Salt Lake City, UT (US); Anindita Roy, Salt Lake City, UT (US)

(21) Appl. No.: 17/715,868

(22) Filed: Apr. 7, 2022

Related U.S. Application Data
(63) Continuation-in-part of application No. 17/715,816, filed on Apr. 7, 2022.
(60) Provisional application No. 63/183,504, filed on May 3, 2021.
(60) Provisional application No. 63/171,761, filed on Apr. 7, 2021.
(60) Provisional application No. 63/240,227, filed on Sep. 2, 2021.
(60) Provisional application No. 63/183,504, filed on May 3, 2021.
(60) Provisional application No. 63/171,761, filed on Apr. 7, 2021.
(60) Provisional application No. 63/240,227, filed on Sep. 2, 2021.

(51) Int. Cl. C12Q 1/6825

(52) U.S. Cl. CPC C12Q 1/6825

(57) ABSTRACT

The present invention relates to electrochemical sensors for oligonucleotide detection. The sensors may feature a probe composition, e.g., an oligonucleotide and an indicator attached to a 5′ end of the probe, attached to a surface such as gold; and a back-filler additive bound to at least a portion space on the surface of the electrochemical sensor not occupied by the probe composition.

### CROSS-REFERENCES TO RELATED APPLICATIONS

This application is a non-provisional and claims benefit of U.S. Provisional Application No. 63/183,504 filed May 3, 2021, the specification of which is incorporated herein in their entirety by reference.

This application is a non-provisional and claims benefit of U.S. Provisional Application No. 63/171,761 filed Apr. 7, 2021 and U.S. Provisional Application No. 63/240,227 filed Sep. 2, 2021, the specification(s) of which is/are incorporated herein in their entirety by reference.

This application is a continuation-in-part and claims benefit of U.S. application Ser. No. 17/715,816 filed Apr. 7, 2022, which claims benefit of U.S. Provisional Application No. 63/183,504 filed May 3, 2021, U.S. Provisional Application No. 63/171,761 filed Apr. 7, 2021 and U.S. Provisional Application No. 63/240,227 filed Sep. 2, 2021, the specifications of which are incorporated herein in their entirety by reference.

### FIELD OF THE INVENTION

The present invention relates to systems and methods to effectively detect diagnostic oligonucleotides from pathogens.

### BACKGROUND OF THE INVENTION

Diagnostic electrochemical sensors, particularly those that can screen and survey for infection in the population, food supply, animals, or the like, have reached a new level of importance. This is because electrochemical sensors allow for a simple, low-cost, sensitive system to measure target oligonucleotide (e.g., DNA or RNA). However, electrochemical sensors still suffer from disadvantages. For example, current electrochemical sensors have unstable gold to sulfur bonds, which led to the early failure of products due to the loss of probes from the gold surface. Additionally, electrochemical sensors have poor and uneven coverage of the probes on the surface, leading to irreproducible results. Finally, probe-to-probe interactions on the surface of the electrochemical sensor lead to drifting signals and inaccurate results. The present invention describes an electrochemical sensor that eliminates these issues.

### BRIEF SUMMARY OF THE INVENTION

It is an objective of the present invention to provide systems, devices, and methods that allow for detecting a diagnostic target oligonucleotide, as specified in the independent claims. Embodiments of the invention are given in the dependent claims. Embodiments of the present invention can be freely combined with each other if they are not mutually exclusive.

In some embodiments, the present inventor features an electrochemical sensor. The electrochemical sensor may comprise a surface, a probe composition (e.g., an oligonucleotide probe) attached to at least a portion of space on the surface, and a back-filler additive bound to at least a portion of space on the surface of the electrochemical sensor not occupied by the probe composition. Said probe composition may comprise an oligonucleotide (e.g., a single-stranded oligonucleotide) and an indicator attached to a 5′ end of the probe. In some embodiments, a 3′ end of the probe is attached to the surface (e.g., via a thiol moiety at the 3′ end). In some embodiments, the back-filler additive comprises a carbon chain and a thiol moiety. In some embodiments, the back-filler additive is bound to the surface via the thiol moiety.

In other embodiments, the present invention may also feature an electrochemical sensor. The electrochemical sensor may comprise a gold surface, a probe composition (e.g., an oligonucleotide probe) attached to at least a portion of space on the gold surface, and a back-filler additive bound to at least a portion space on the gold surface not occupied by the probe composition. The probe composition may comprise an oligonucleotide (e.g., a single-stranded oligonucleotide) and an indicator attached to a 5′ end of the probe. In some embodiments, a 3′ end of the probe is attached to the gold surface (e.g., via a thiol moiety at the 3′ end). In some embodiments, the back-filler additive comprises a carbon chain and a thiol moiety, In some embodiments, the back-filler additive is bound to the surface via the thiol moiety.

One of the unique and inventive technical features of the present invention is an electrochemical sensor comprising evenly spaced probes stably bonded to the electrochemical surface. Without wishing to limit the invention to any theory or mechanism, it is believed that the technical feature of the present invention advantageously provides for accurate and reproducible results when the electrochemical sensor is used. None of the presently known prior references or work has the unique, inventive technical feature of the present invention.

The unique and inventive technical features of the electrochemical sensors described herein are acquired through the methods used to manufacture said sensors. For example, the electrochemical sensors are manufactured using a two-step method to attach the DNA probe to the electrochemical sensor (e.g., the DNA is put onto the surface of the electrochemical sensor first, and then a reducing agent is added afterward to “glue” the DNA probe to the surface of the electrochemical sensor). By adding the reducing agent (e.g., TCEP) in a separate step (e.g., after the DNA probe is put onto the surface of the electrochemical sensor), competitive binding between the DNA probe and the reducing agent for the surface of the electrochemical sensor is eliminated.

Additionally, the electrochemical sensors described herein undergo an electrochemical cleaning to prepare the surface of the electrochemical sensor (e.g., a gold surface). In some embodiments, the electrochemical cleaning increases the strength of the gold-sulfur bond by di-thiol reduction with a reducing agent (e.g., TCEP) after the oligonucleotides (e.g., DNA probes) adhere to the surface. In some embodiments, electrochemically cleaning the surface of the electrochemical sensor (e.g., a gold surface) increases the amount of oligonucleotides (e.g., DNA probes) bound to the surface of the gold compared to an unclean surface. By increasing the bond strength between the gold surface and the oligonucleotides (i.e., the gold surface and the sulfur at the 3′ end of the oligonucleotides (e.g., the DNA probes)), the present invention is able to lower the concentration of oligonucleotides (e.g., DNA probes) on the surface of the electrochemical sensor (e.g., a gold surface) while still maintaining signal strength. This allows for more uniform coverage of the surface of the electrochemical sensor (e.g., a gold surface).

Lastly, the electrochemical sensors described herein undergo clean-up steps to break interactions between the terminated oligonucleotides (e.g., DNA probes) and stabilize the electrochemical sensor. The clean-up steps may comprise adding an additive (e.g., a buffer or a salt solution) to disrupt probe-to-probe interactions before using the electrochemical sensor. This allows for more reproducible and stable signals without shifting backgrounds, such that more reproducible data is obtained with lower detection and sensitivity.

Furthermore, the prior references teach away from the present invention. For example, prior references teach mixing the DNA probe and the reducing agent together and allowing that mixture to react first before adding it to the electrochemical sensor surface, which causes a reduction in the probe coverage on the surface. Specifically, the reducing agent competes with the DNA probe to bind to the electrochemical surface (e.g., a gold surface). Therefore, when the DNA probe is mixed with the reducing agent and put onto the surface together, there is a competition between the DNA probe and the reducing agent for any available binding sites on the surface of the electrochemical sensor. Thus, instead of getting maximum coverage of the DNA probe on the surface of the electrochemical sensor, a mixed coverage between the DNA probe and the reducing agent is observed.

For more details of such methods, the specification of U.S. application Ser. No. 17/715,816 is hereby incorporated in its entirety by reference.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a method for producing an electrochemical sensor, in accordance with some embodiments described herein.

FIGS. 2A and 2B show methods for mixing disulfide terminated oligonucleotides (e.g., oligonucleotide probes) with a gold substrate. FIG. 2A shows a 40 μL drop of a DNA solution (e.g., oligonucleotide probes and a solvent) placed on top of the electrode. FIG. 2B shows the electrochemical sensor submerged in a DNA solution (e.g., oligonucleotide probes and a solvent). For both methods described in FIGS. 2A and 2B, the electrochemical sensor was allowed to sit with the DNA solution for about 30 minutes before a composition for reducing thiol moieties was added.

FIG. 3 shows, in accordance with some embodiments, an electrochemical sensor as described herein.

FIGS. 4A and 4B show the finished electrochemical sensor. FIG. 4A shows a glass test strip. The center circle shows an electrochemical sensor as described herein; the curved arc on top of the circle is the counter electrode. The small electrode on the right is the reference electrode. The probe is only found on the center circle. FIG. 4B shows a gold disk electrode supported by a PEEK material (polyetheretherketone) substrate. This is the finished product for the working electrode (sensor). Counter and Reference not shown.

FIG. 5 shows, in accordance with some embodiments, how the electrochemical sensor technology works when a target oligonucleotide binds to a probe.

FIGS. 6A and 6B show the setup for using the electrochemical sensor described herein. FIG. BA shows a three-electrode setup; the counter electrode is shown with a black lead, the reference electrode is shown with a blue lead, and the working electrode (i.e., the electrode comprising the electrochemical sensor described herein) is shown with a red lead. All electrodes comprising oligonucleotide probes on the surface were placed in a 10 mM PBS solution pH 7.4.

FIG. 7 shows an atomic force micrograph of the gold laminated glass test strips. The image area was five microns by five microns. The color scale was set to fifty nanometers. Peak to peak surface roughness was approximately 600 pm with exclusions due to contamination which was around 80 nm.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

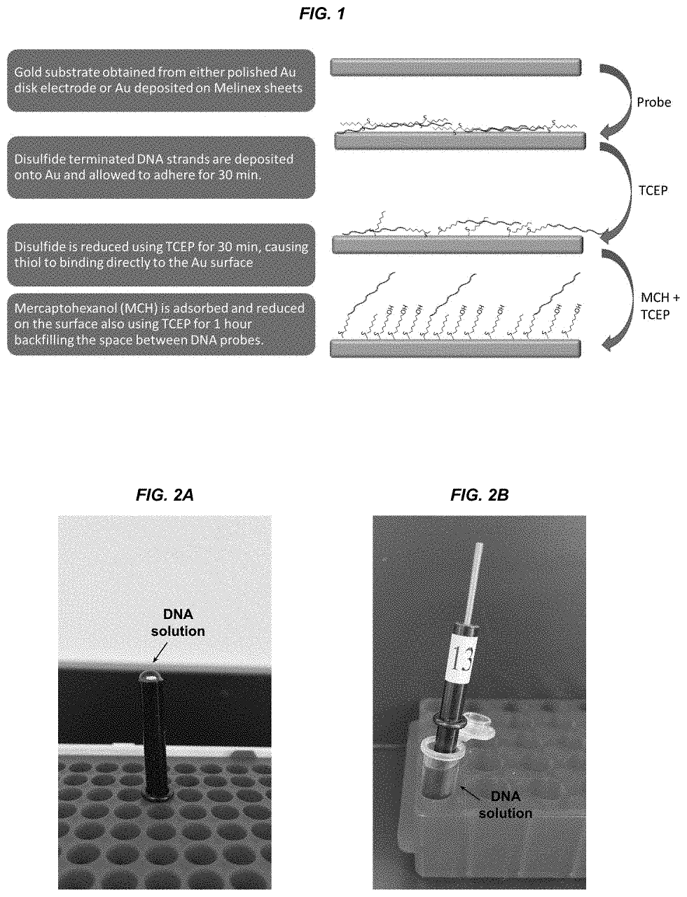

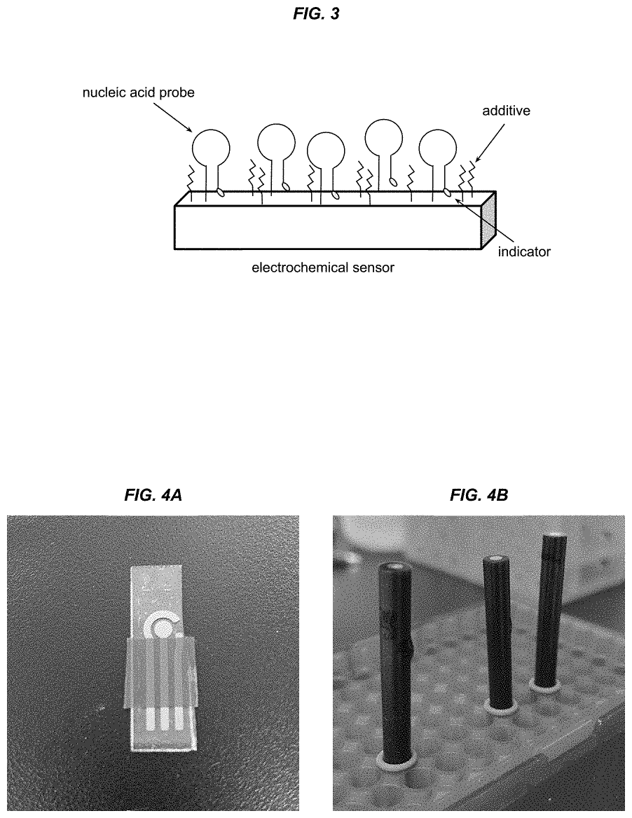

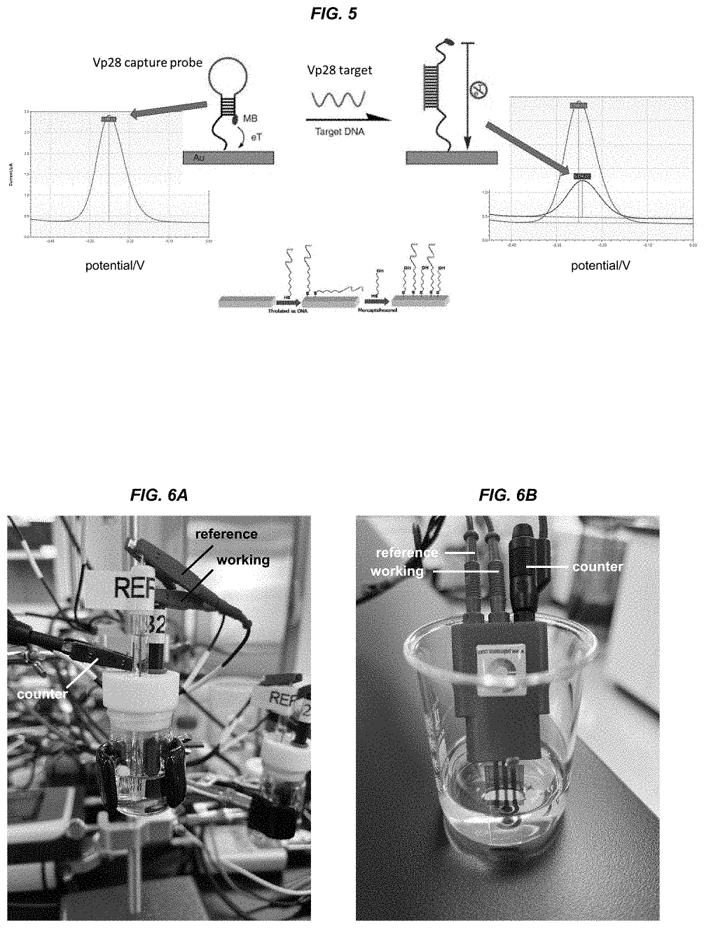

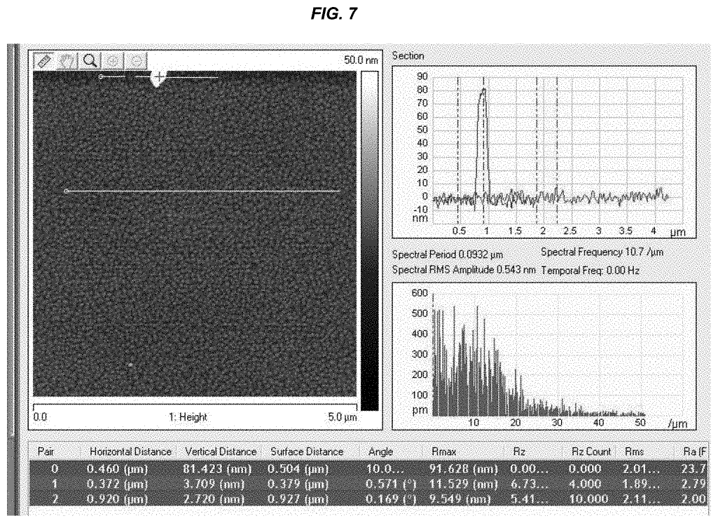

Referring now to FIG. 1-7, the present invention features systems. devices, and methods that allow for point-of-care diagnostics that allow for the identification of various pathogens.

The present invention features an electrochemical sensor comprising a surface (e.g., a gold surface), a probe composition attached to at least a portion of space on the surface, and a back-filler additive bound to at least a portion of space on the surface of the electrochemical sensor not occupied by the probe composition. In some embodiments, said probe composition comprises an oligonucleotide (e.g., a single-stranded oligonucleotide) and an indicator attached to a 5′ end of the probe.

The present invention may also feature an electrochemical sensor comprising a gold surface, a probe composition attached to at least a portion of space on the gold surface, and a back-filler additive bound to at least a portion of space on the gold surface not occupied by the probe composition. In some embodiments, said probe composition comprises a single stranded oligonucleotide and an indicator attached to a 5′ end of the probe. In some embodiments, a 3′ end of the probe is attached to the gold surface (e.g., via a free thiol moiety at a 3′ end). In some embodiments, the back-filler additive comprises a carbon chain and a thiol moiety. In some embodiments, the back-filler additive is bound to the surface via the thiol moiety.

The present invention may further feature an electrochemical sensor comprising a gold surface, a probe composition attached to at least a portion of space on the gold surface, and a back-filler additive bound to at least a portion of space on the gold surface not occupied by the probe composition. In some embodiments, said probe composition comprises a single stranded oligonucleotide which is a reverse complement of a target oligonucleotide and an indicator attached to a 5′ end of the probe. In some embodiments, a 3′ end of the probe is attached to the gold surface (e.g., via a free thiol moiety at a 3′ end). In some embodiments, the back-filler additive comprises a carbon chain and a thiol moiety. In some embodiments, the back-filler additive is bound to the surface via the thiol moiety.

In some embodiments, the oligonucleotide comprises DNA or RNA. In some embodiments, the oligonucleotide is single-stranded. In some embodiments, the oligonucleotide comprises single-stranded DNA (ssDNA) or single-stranded RNA (ssRNA). In some embodiments, the oligonucleotide of the probe composition is a reverse complement of a target oligonucleotide. The target oligonucleotide may be a single-stranded target oligonucleotide or a double-stranded target oligonucleotide.

In some embodiments, the probe composition is attached to at least a portion of space on the surface electrochemical sensor by first mixing the probe composition having a free thiol moiety at a 3′ end with the substrate (e.g., gold substrate). Subsequently, a composition for reducing thiol moieties of the oligonucleotides (e.g., the probe composition) is introduced to the substrate (e.g., the electrochemical sensor, e.g., gold substrate), thereby causing the oligonucleotides to bind a surface of the gold substrate.

In some embodiments, the oligonucleotides comprising a free thiol moiety at a 3′ end are disulfide terminated oligonucleotides. In preferred embodiments, the disulfide terminated oligonucleotides (e.g., the oligonucleotide probes) are mixed with the gold substrate 30 minutes before the probes are chemically bound using a reducing agent (e.g., TCEP). In other embodiments, the disulfide terminated oligonucleotides (e.g., the oligonucleotide probes) are mixed with the gold substrate for about 15 to 240 minutes, or about 15 to 210 minutes, or about 15 to 180 minutes, or about 15 to 150 minutes, or about 15 to 120 minutes, or about 15 to 90 minutes, or about 15 to 60 minutes, or about 15 to 30 minutes, or about 30 to 240 minutes, or about 30 to 210 minutes, or about 30 to 180 minutes, or about 30 to 150 minutes, or about 30 to 120 minutes, or about 30 to 90 minutes, or about 30 to 60 minutes, or about 60 to 240 minutes, or about 60 to 210 minutes, or about 60 to 180 minutes, or about 60 to 150 minutes, or about 60 to 120 minutes, or about 60 to 90 minutes, or about 90 to 240 minutes, or about 90 to 210 minutes, or about 90 to 180 minutes, or about 90 to 150 minutes, or about 90 to 120 minutes, or about 120 to 240 minutes, or about 120 to 210 minutes, or about 120 to 180 minutes, or about 120 to 150 minutes, or about 150 to 240 minutes, or about 150 to 210 minutes, or about 150 to 180 minutes, or about 180 to 240 minutes, or about 180 to 210 minutes, or about 210 to 240 minutes, before the probes are chemically bound using a reducing agent (e.g., TCEP). In some embodiments, the disulfide terminated oligonucleotides (e.g., the probes) are mixed with the gold substrate for about 15 minutes, or about 30 minutes, or about 60 minutes, or about 75 minutes, or about 90 minutes, or about 105 minutes, or about 120 minutes, or about 135 minutes, or about 150 minutes, or about 165 minutes, or about 180 minutes, or about 195 minutes, or about 210 minutes, or about 225 minutes, or about 240 minutes before the probes are chemically bound using a reducing agent (e.g., TCEP).

Excess thiol and probe composition may be removed. In some embodiments, the excess thiol and probe composition are physically removed, e.g., by flicking the electrochemical sensor. In other embodiments, the excess thiol and probe composition are removed by wicking the solution off, e.g., by using a tissue to wick the solution off.

In preferred embodiments, the reducing agent (e.g., TCEP) is added to the gold substrate comprising disulfide terminated oligonucleotides (e.g., probes) for 30 to 60 minutes. In some embodiments, the reducing agent (e.g., TCEP) is added to the gold substrate comprising disulfide terminated oligonucleotides (e.g., probes) for about 15 to 120 minutes, or about 15 to 90 minutes, or about 15 to 60 minutes, or about 15 to 30 minutes, or about 30 to 120 minutes or about 30 to 90 minutes, or about 30 to 60 minutes, or about 60 to 120 minutes, or about 60 to 90 minutes, or about 90 to 120 minutes. In some embodiments, the reducing agent (e.g., TCEP) is added to the gold substrate comprising disulfide terminated oligonucleotides (e.g., probes) overnight (e.g., for about 12 to 16 hours). In other embodiments, the reducing agent (e.g., TCEP) is added to the gold substrate comprising disulfide terminated oligonucleotides (e.g., probes) for a minimum of about 5 minutes. In some embodiments, the reducing agent (e.g., TCEP) is added to the gold substrate comprising disulfide terminated oligonucleotides (e.g., probes) for about 15 minutes, or about 30 minutes, or about 60 minutes, or about 90 minutes, or about 120 minutes.

The back-filler additive is then added and allowed to form a covalent bond directly to at least a portion of space on the surface of the electrochemical sensor not occupied by the probe composition. In some embodiments, the back-filler additive is a molecule that already has a sulfur terminated end and that naturally binds to the surface of the electrochemical sensor (e.g., gold) not occupied by the probe composition (e.g., exposed gold). The back-filler additive naturally forms a self-assembled monolayer. In some embodiments, the back-filler additive is added to the surface of the gold substrate without any other additions. In other embodiments, the back-filler additive is added to the surface of the gold substrate with a reducing agent (e.g., TCEP). Without wishing to limit the present invention to any theory or mechanism, it is believed that adding a reducing agent (e.g., TCEP) along with the back-filler addictive to the surface of the gold substrate increases the strength of the sulfur-gold bond by guaranteeing that all of the sulfur has been fully reduced.

As used herein, an “indicator” may refer to an electrochemically active molecule that can be covalently attached to the probe composition. In some embodiments, the indicator transfers electrons to and from the surface of an electrochemical sensor described herein, creating a voltage-dependent current that can be measured with a potentiostat. The indicator may comprise methylene blue, methylene violet, Ruthenium hexamine, or ferrocene. Other indicators may be used in accordance with compositions and methods as described herein.

The voltage-dependent current may be scanned using a square wave voltammetry. In some embodiments, the square wave voltammeter is set up using a 3 electrode setup; with the electrochemical sensor described herein (i.e., DNA/electrode) being the working electrode, the 3 M KCl/AgCl/Ag being the reference electrode, and a Platinum disk or gold substrate as the counter counter electrode.

In some embodiments, the target oligonucleotide is DNA or RNA (e.g., mRNA). In some embodiments, the target oligonucleotide is from a pathogen. In some embodiments, the pathogen is a virus, bacteria, fungi, or prion. In some embodiments, the virus is the white spot syndrome virus. In some embodiments, the pathogen is a sexually transmitted disease, including but limited to Herpes Simplex Virus, Syphillis, Gonorrhea, Trichomonas, and Chlamydia. In other embodiments, the pathogen is Influenza or a variant thereof (e.g., H1N1 and H5N1), or Covid-19.

In some embodiments, the electrochemical sensors described herein detect a target oligonucleotide sequence for a white spot syndrome virus envelope protein. In some embodiments, the envelope protein is VP24, VP26, VP28, or a combination thereof.

As used herein, a “back-filler addictive” refers to additional molecules which bind onto the surface of the electrochemical sensor (e.g., gold) to fill any space on the surface of the electrochemical sensor not occupied by the probe composition described herein. Such molecules include, but are not limited to, hydrocarbon chains comprising at least two terminal thiol groups (e.g., hexane di-thiol). In other embodiments, additives may include molecules comprising internal di-sulfide groups that can be reduced to form a two-terminal thiol group.

In some embodiments, the back-filler additive is organic. In some embodiments, the back-filler additive comprises a thiol moiety at a first end. In some embodiments, the thiol moiety binds to the surface of the electrochemical sensor (e.g., the surface of a gold substrate). In some embodiments, the back-filler additive further comprises a carbon chain linked to the thiol moiety at the first end of the back-filler additive. In some embodiments, the carbon chain is a hydrocarbon chain. In some embodiments, the carbon chain is linear. In other embodiments, the carbon chain is branched. In some embodiments, the back-filler additive comprises a hydrocarbon chain linked to a thiol moiety. In further embodiments, the back-filler additive is nonreactive. In certain embodiments, the back-filler additive is mercaptohexanol.

The carbon chain of the back-filler additive may help to physically separate the surface of the electrochemical sensor (e.g., the gold surface) from the solution. In some embodiments, the carbon chain is a hydrocarbon chain. In preferred embodiments, the hydrocarbon chain comprises a six hydrocarbon chain. In some embodiments, the hydrocarbon chain comprises a chain of about four hydrocarbons, or about five hydrocarbons, or about six hydrocarbons, or about seven hydrocarbons, or about eight hydrocarbons. Without wishing to limit the present invention to any theories or mechanisms it is believed that the carbon chain (e.g., the hydrocarbon chain) of the back-filler additive should be long enough to prevent other ions from getting close to the surface of the electrochemical sensor (e.g., the gold surface) which may cause additional electrochemical reactions, while not being too long to prevent the indicator (e.g., Methylene Blue) from reacting. In some embodiments, back-filler additives with shorter carbon chains (e.g., a three hydrocarbon chain) result in additional noise. In other embodiments, back-filler additives with longer carbon chains (e.g., a nine hydrocarbon chain) results in no signal from the desired reaction

In some embodiments, the electrochemical sensors described herein are adapted to differentiate between hybridization rates of a target oligonucleotide and a probe composition bound to the sensor surface.

The present invention features an electrochemical sensor comprising a probe attached to a surface of the electrochemical sensor and a back-filler additive. In some embodiments, the back-filler additive fills any space on the surface of the electrochemical sensor not occupied by the probe.

The present invention may further feature an electrochemical sensor comprising a gold surface, a probe composition attached to at least a portion of space on the surface, and a back-filler additive bound to at least a portion of space on the surface of the electrochemical sensor not occupied by the probe composition. The probe composition may comprise a single-stranded oligonucleotide which is a reverse complement of a target oligonucleotide and an indicator attached to a 5′ end of the probe, the 3′ end being attached to the surface. The back-filler additive may comprise a carbon chain and a thiol moiety, and the additive is bound to the surface via the thiol moiety.

In some embodiments, the electrochemical sensors described herein are able to detect a single stranded target oligonucleotide sequence that is complementary to the probe attached to a said sensor. For example, in some embodiments, the target oligonucleotide is a DNA oligonucleotide or an RNA oligonucleotide. In some embodiments, the electrochemical sensors described herein are able to detect a double-stranded target oligonucleotide sequence that is complementary to the probe attached to a said sensor.

The present invention features a method of detecting a target oligonucleotide (e.g., a single-stranded target oligonucleotide). The method comprises adding a sample to an electrochemical sensor as described herein, the sample comprising the target oligonucleotide (e.g., a single stranded target oligonucleotide) to the electrochemical sensor, and measuring a loss of current on the electrochemical sensor. In some embodiments, the loss of current is proportional to the amount of the single-strand target oligonucleotides bound to the probe.

In some embodiments, the present invention features a method of detecting a double-stranded target oligonucleotide (e.g., a dsDNA target) using the electrochemical sensors as described herein. The method may comprise heating the double-stranded target oligonucleotide above its melting temperature (e.g., heating to 70° C.; to denature the double-stranded target oligonucleotide into single strands) and adding the target oligonucleotide to the electrochemical sensor. Once the target oligonucleotide is added to the electrochemical sensor, the target oligonucleotide is cooled to below its melting temperature to allow the target oligonucleotides to anneal with the probe compositions on the surface of the electrochemical sensor. The method comprises measuring a loss of current on the electrochemical sensor. In some embodiments, the loss of current is proportional to the amount of the single-strand target oligonucleotides bound to the probe.

In some embodiments, when no target oligonucleotide is bound to the probe composition, the indicator is available to interact with the surface of the electrochemical sensor (e.g., a gold surface), and a larger current peak is seen. In other embodiments, when a target oligonucleotide is bound to the probe composition, the indicator is no longer available to interact with the surface of the electrochemical sensor (e.g., a gold surface), thus decreasing the measured current. See FIG. 5 for an example.

Square wave voltammetry may be used to measure the reaction between the indicator and the surface of the electrochemical sensor (e.g., gold). In some embodiments, the indicator transfers electrons to and from the surface of the electrochemical surface, creating a voltage-dependent current that can be measured with a potentiostat. The potentiostat is the equipment that records the current that is generated by the voltage at the electrodes (i.e., what is plotted by a computer) and controls the signals generated/used to perform square wave voltammetry.

In some embodiments, the setup comprises a potentiostat and one or more electrodes. In other embodiments, the setup comprises a potentiostat and two or more electrodes. In further embodiments, the setup comprises a potentiostat and one electrode, or two electrodes, or three electrodes, or four electrodes, or more electrodes. In some embodiments, the setup comprises a potentiostat, an electrochemical sensor as described herein (i.e., a working electrode), and a reference electrode. In other embodiments, the setup comprises a potentiostat, an electrochemical sensor as described herein (i.e., a working electrode), a reference electrode, and a counter electrode.

As used herein, a “reference electrode” refers to an electrode that has a stable and well-known electrode potential. Non-limiting examples of reference electrodes may include but are not limited to stable reference electrodes such as a calomel electrode or a quasi-reference electrode like a chloridized silver wire. In preferred embodiments, the reference electrode comprises a 3M KCl/AgCl/Ag reference electrode. As used herein, a “counter electrode” or an “auxiliary electrode” may be used interchangeably and refers to an electrode that is used to close the current circuit in the electrochemical cell and does not participate in the electrochemical reaction. Non-limiting examples of counter electrodes include but are not limited to an unmodified Platinum electrode or an unmodified gold electrode.

### EXAMPLE 1

The following is a non-limiting example of the present invention. It is to be understood that said example is not intended to limit the present invention in any way. Equivalents or substitutes are within the scope of the present invention.

As described above, an indicator is an electrochemically active molecule such as Methylene Blue, that transfers electrons to and from the surface of the electrochemical surface (e.g., a gold surface) to create a voltage-dependent current that can be measured with a potentiostat. The voltage is scanned using square wave voltammetry. The voltages are started approximately 200 mV lower than its standard redox potential and are scanned positive until a final potential approximately 200 mV higher than the standard redox potential is reached.

The voltages are started at lower than the oxidation/reduction (redox) potential because at this potential, no reaction occurs. Square wave voltammetry measures small current differences in an alternating voltage. At potentials well below the redox potential, these current differences are small. Then the voltage is scanned in the positive direction. As the voltage approaches, the oxidation/reduction potential of the indicator (i.g., the redox molecule; e.g., methylene blue) will start to oxidize. The differences in current between points generated by the alternating voltage grow to their maximum near the redox equilibrium potential, giving a peak current in the square wave voltammetry experiments. As the voltage is scanned to well above the redox potential, the reaction becomes self-limiting and the differences in current caused by the alternating voltage drop to a minimum again.

### EMBODIMENTS

The following embodiments are intended to be illustrative only and not to be limiting in any way.

Embodiment 1: An electrochemical sensor comprising: (a) a surface; (b) a probe composition attached to at least a portion of space on the surface, said probe composition comprising: an oligonucleotide and an indicator attached to a 5′ end of the probe; and (c) a back-filler additive bound to at least a portion of space on the surface of the electrochemical sensor not occupied by the probe composition.

Embodiment 2: The sensor of embodiment 1, wherein the surface is gold.

Embodiment 3: The sensor of any one of embodiments 1-2, wherein the oligonucleotide comprises DNA or RNA.

Embodiment 4: The sensor of any one of embodiments 1-3, wherein the oligonucleotide is single stranded.

Embodiment 5: The sensor of any one of embodiments 1-4, wherein the oligonucleotide of the probe composition is a reverse complement of a target oligonucleotide.

Embodiment 6: The sensor of embodiment 5, wherein the target oligonucleotide is a single stranded target oligonucleotide.

Embodiment 7: The sensor of embodiment 5 or 6, wherein the target oligonucleotide is from a pathogen.

Embodiment 8: The sensor of embodiment 7, wherein the pathogen is a sexually-transmitted disease.

Embodiment 9: The sensor of embodiment 7, wherein the pathogen is a virus, bacteria, fungi, or prion.

Embodiment 10: The sensor of embodiment 8, wherein the virus is a white spot syndrome virus.

Embodiment 11: The sensor of embodiment 8, wherein the virus is a SARS-CoV-2,

Embodiment 12: The sensor of any of embodiments 1-11, wherein the back-filler additive is organic.

Embodiment 13: The sensor of any of embodiments 1-11, wherein the back-filler additive comprises a thiol moiety at a first end.

Embodiment 14: The sensor of embodiment 13, wherein the thiol moiety binds to the surface of the electrochemical sensor.

Embodiment 15: The sensor of embodiment 13 or embodiment 14, wherein the back-filler additive further comprises a carbon chain linked to the thiol moiety at the first end of the back-filler additive.

Embodiment 16: The sensor of embodiment 15, wherein the carbon chain is a hydrocarbon chain.

Embodiment 17: The sensor of embodiment 15 or embodiment 16, wherein the carbon chain is linear.

Embodiment 18: The sensor of embodiment 15 or embodiment 16, wherein the carbon chain is branched.

Embodiment 19: The sensor of embodiment 13. wherein the back-filler additive comprises a hydrocarbon chain linked to the thiol moiety.

Embodiment 20: The sensor of any one of embodiments 1-19, wherein the back-filler additive is nonreactive.

Embodiment 21: The sensor of any of embodiments 1-11, wherein the back-filler additive is mercaptohexanol.

Embodiment 22: The electrochemical sensor of any one of embodiments 1-21, wherein the sensor is adapted to differentiate between hybridization rates of a target oligonucleotide and a probe composition bound to the sensor surface.

Embodiment 23; An electrochemical sensor comprising: (a) a gold surface; (b) a probe composition attached to at least a portion of space on the gold surface, said probe composition comprising: a single stranded oligonucleotide and an indicator attached to a 5′ end of the probe, wherein a 3′ end is attached to the gold surface; and (c) a back-filler additive bound to at least a portion of space on the gold surface of the electrochemical sensor not occupied by the probe composition, the back-filler additive comprises a carbon chain and a thiol moiety, and the back-filler additive is bound to the surface via the thiol moiety.

Embodiment 24: The sensor of embodiment 23, wherein the single stranded oligonucleotide is a reverse complement of a target oligonucleotide.

Embodiment 25: An electrochemical sensor comprising: (a) a gold surface; (b) a probe composition attached to at least a portion of space on the gold surface_{;} said probe composition comprising: a single stranded oligonucleotide which is a reverse complement of a target oligonucleotide and an indicator attached to a 5′ end of the probe, the 3′ end being attached to the surface; and (c) a back-filler additive bound to at least a portion of space on the gold surface of the electrochemical sensor not occupied by the probe composition, the back-filler additive comprises a carbon chain and a thiol moiety, and the additive is bound to the surface via the thiol moiety.

Embodiment 26: A method of detecting a target oligonucleotide, the method comprising: (a) adding a sample to an electrochemical sensor according to any one of embodiments 1-25, the sample comprising the target oligonucleotide; and (b) measuring a loss of current on the electrochemical sensor, wherein the loss of current is proportional to the amount of the target oligonucleotide bound to the probe.

Embodiment 27: The method of embodiment 26, wherein the target oligonucleotide is a single-stranded target oligonucleotide.

Embodiment 28: The method of embodiment 26, wherein the target oligonucleotide is a double-stranded target oligonucleotide.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An electrochemical sensor comprising:
a) a surface;
b) a probe composition attached to at least a portion of space on the surface, said probe composition comprising: an oligonucleotide and an indicator attached to a 5′ end of the probe; and
c) a back-filler additive bound to at least a portion of space on the surface of the electrochemical sensor not occupied by the probe composition.

2. The sensor of claim 1, wherein the surface is gold.

3. The sensor of claim 1, wherein the oligonucleotide of the probe composition comprises DNA or RNA.

4. The sensor of claim 1, wherein the oligonucleotide of the probe composition is single stranded.

5. The sensor of claim 1, wherein the oligonucleotide of the probe composition is a reverse complement of a target oligonucleotide.

6. The sensor of claim 1, wherein the back-filler additive is organic.

7. The sensor of claim 1, wherein the back-filler additive is nonreactive.

8. The sensor of claim 1, wherein the back-filler additive is mercaptohexanol.

9. The sensor of claim 1, wherein the back-filler additive comprises a thiol moiety, the thiol moiety is bound to the surface of the electrochemical sensor.

10. The sensor of claim 9, wherein the back-filler additive further comprises a carbon chain linked to the thiol moiety at a first end.

11. An electrochemical sensor comprising:
a) a gold surface;
b) a probe composition attached to at least a portion of space on the gold surface, said probe composition comprising: a single stranded oligonucleotide and an indicator attached to a 5′ end of the probe, wherein a 3′ end is attached to the gold surface; and
c) a back-filler additive bound to at least a portion of space on the gold surface of the electrochemical sensor not occupied by the probe composition, the back-filler additive comprises a carbon chain and a thiol moiety, and the back-filler additive is bound to the surface via the thiol moiety.

12. The sensor of claim 11, wherein the oligonucleotide is single stranded.

13. The sensor of claim 11, wherein the oligonucleotide of the probe composition is a reverse complement of a target oligonucleotide.

14. The sensor of claim 11, wherein the back-filler additive is organic.

15. The sensor of claim 11, wherein the back-filler additive is nonreactive.

16. The sensor of claim 11, wherein the back-filler additive is mercaptohexanol.

17. An electrochemical sensor comprising:
a) a gold surface;
b) a probe composition attached to at least a portion of space on the gold surface, said probe composition comprising: a single stranded oligonucleotide and an indicator attached to a 5′ end of the probe, the 3′ end being attached to the surface; and
c) a back-filler additive bound to at least a portion of space on the gold surface of the electrochemical sensor not occupied by the probe composition, the back-filler additive comprises a carbon chain and a thiol moiety, and the additive is bound to the surface via the thiol moiety.

18. The sensor of claim 17, wherein the single stranded oligonucleotide of the probe composition is a reverse complement of a target oligonucleotide.

19. The sensor of claim 17, wherein the back-filler additive is organic.

20. The sensor of claim 17, wherein the back-filler additive is nonreactive.
